(19) United States
(12) Patent Application Publication
(10) Pub. No.: US 2021/0330177 A1
(43) Pub. Date: Oct. 28, 2021

(54) ENDOSCOPE

(71) Applicant: PANASONIC I-PRO SENSING SOLUTIONS CO., LTD., Fukuoka (JP)

(72) Inventors: Haruhiko KOHNO, Fukuoka (JP); Risa KOMATSU, Fukuoka (JP)

(73) Assignee: PANASONIC I-PRO SENSING SOLUTIONS CO., LTD., Fukuoka (JP)

(21) Appl. No.: 17/369,444

(22) Filed: Jul. 7, 2021

Related U.S. Application Data
(63) Continuation of application No. PCT/JP2019/038046, filed on Sep. 26, 2019.

(30) Foreign Application Priority Data
Jan. 9, 2019 (JP) 2019-002004

(51) Int. Cl. A61B 1/00; A61B 1/05

(52) U.S. Cl. CPC A61B 1/00197; A61B 1/00096; A61B 1/00174; A61B 1/00018; A61B 1/00193; A61B 1/05

(57) ABSTRACT

This endoscope is provided with: a hard part that is provided at the distal end of a scope, is formed in a substantially cylindrical shape, and has a substantially circular distal end surface; and a plurality of cameras that are disposed on the left and right sides of the hard part to be located on both sides of a first virtual line perpendicular to the axis of the hard part on the distal end surface. The plurality of cameras include a first camera. The first camera is disposed so that the imaging axis is offset in the direction along the first virtual line from a second virtual line perpendicular to the axis and the first virtual line.

### CROSS-REFERENCE TO RELATED APPLICATIONS

This is a continuation of International Application No. PCT/JP2019/038046 filed on Sep. 26, 2019, and claims priority from Japanese Patent Application No. 2019-002004 filed on Jan. 9, 2019, the entire content of which is incorporated herein by reference.

### TECHNICAL FIELD

The present disclosure relates to an endoscope.

### BACKGROUND ART

An endoscope for reducing the size of an insertion distal end portion has been proposed (for example, refer to PTL 1). The endoscope has an imaging device including a prism in which a first prism and a second prism are joined such that incident light that has passed through an objective optical system is split into two optical paths and emitted, a first solid-state imaging element that receives light reflected by a junction surface between the first prism and the second prism and emitted from the prism, and a second solid-state imaging element that receives light transmitted through the first and second prisms and emitted from the prism. The endoscope includes: a first connection portion that is provided on one side surface side of the first solid-state imaging element and connects the first solid-state imaging element and a first substrate; and a second connection portion that is provided on one side surface side of the second solid-state imaging element and connects the second solid-state imaging element and a second substrate. The first solid-state imaging element and the second solid-state imaging element are disposed such that side surfaces of the first solid-state imaging element and the second solid-state imaging element which do not have the first connecting portion and the second connecting portion are close to each other and face each other.

### CITATION LIST

### Patent Literature

[PTL 1]: JP-A-2008-99746

### SUMMARY OF INVENTION

### Technical Problem

However, in an endoscope that obtains an image such as a stereoscopic vision from a parallax of two or more eyes, it is necessary to transmit an image signal obtained by an imaging optical system to an external device such as a video processor for generating a stereoscopic image while minimizing an increase in the outer diameter of the insertion distal end portion. In particular, when a multi-eye endoscope is realized by mounting an image sensor on the insertion distal end portion, a space for arranging a cable for transmitting an image signal from an image sensor is necessary for the insertion distal end portion, which causes an increase in the outer diameter of the endoscope. However, the above-mentioned PTL 1 does not consider a technical measure for solving this cause.

The present disclosure has been made in view of the related situation described above, and an object of the present disclosure is to provide an endoscope capable of suppressing an increase in an outer diameter in an endoscope of two or more eyes.

### Solution to Problem

According to an aspect of the present disclosure, there is provided an endoscope including: a rigid portion provided at a distal end of a scope and having a substantially cylindrical distal end surface formed in a substantially cylindrical shape; and a plurality of cameras disposed on left and right sides of the rigid portion sandwiching a first virtual line orthogonal to an axis line of the rigid portion on the distal end surface, in which the plurality of cameras include a first camera, and the first camera is disposed such that an imaging axis is shifted from a second virtual line orthogonal to each of the axial line and the first virtual line in a direction along the first virtual line.

### Advantageous Effects of Invention

According to the present disclosure, it is possible to suppress an increase in outer diameter in the endoscope with two or more eyes.

### BRIEF DESCRIPTION OF DRAWINGS

FIG. 1 is a view showing an example of an outline of an endoscope system according to a first embodiment;

FIG. 2 is a perspective view showing an appearance of a distal end of the scope;

FIG. 3 is a longitudinal cross sectional view showing a configuration of a right eye camera disposed in the scope;

FIG. 4 is a front view of a rigid portion as viewed from a subject side;

FIG. 5 is a block diagram showing a hardware configuration example of the endoscope system according to the first embodiment;

FIG. 6 is a front view of a first modification of the endoscope according to the first embodiment;

FIG. 7 is a front view of a second modification of the endoscope according to the first embodiment;

FIG. 8 is a front view of a third modification of the endoscope according to the first embodiment;

FIG. 9 is a front view of a fourth modification of the endoscope according to the first embodiment;

FIG. 10 is a front view of a fifth modification of the endoscope according to the first embodiment;

FIG. 11 is a front view of a sixth modification of the endoscope according to the first embodiment.

### DESCRIPTION OF EMBODIMENTS

Hereinafter, embodiments in which the configuration and function of an endoscope according to the present disclosure are specifically disclosed will be described in detail with reference to the drawings as appropriate. However, an unnecessarily detailed description may be omitted. For example, a detailed description of a well-known matter or a repeated description of substantially the same configuration may be omitted. This is to avoid unnecessary redundancy in the following description and to facilitate understanding of those skilled in the art. The accompanying drawings and the following description are provided for a thorough understanding of the present disclosure for those skilled in the art, and are not intended to limit the subject matter in the claims.

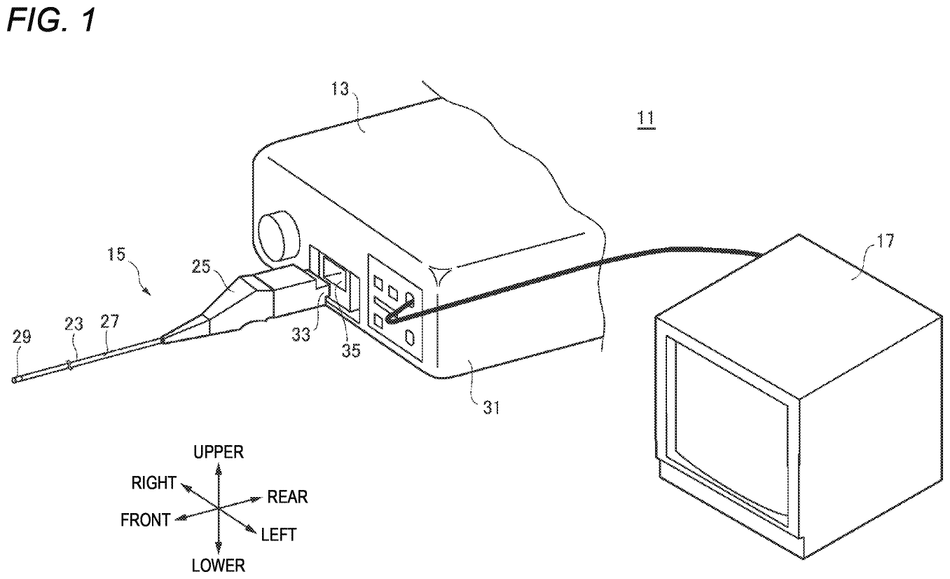

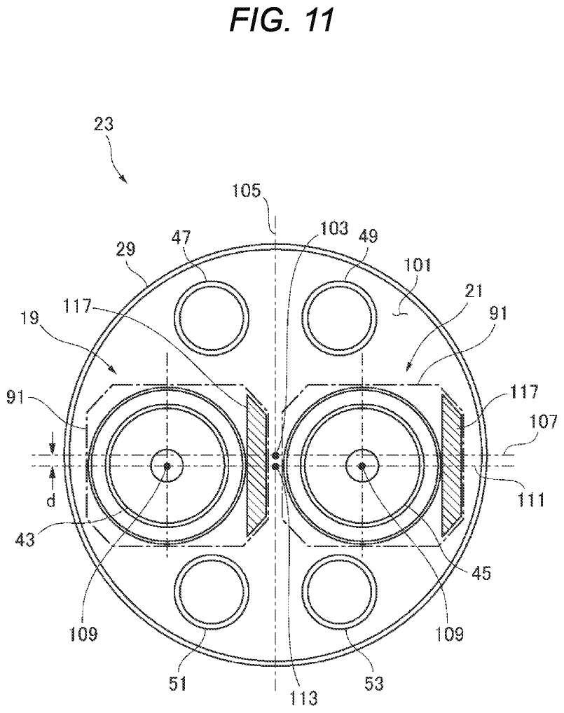

FIG. 11 is a view illustrating an example of an outline of an endoscope system 11 according to a first embodiment. In the following description, “upper”, “lower”, “right”, “left”, “front”, and “rear” follow the respective directions shown in FIG. 1. For example, the upper direction and the lower direction of a video processor 13 placed on the horizontal plane are referred to as “upper” and “lower”, respectively, the side where an endoscope 15 images an observation target is referred to as “front”, and the side where the endoscope 15 is connected to the video processor 13 is referred to as “rear”. Further, facing forward from the distal end of the endoscope 15 in an insertion direction, the right hand side corresponds to “right”, and the left hand side corresponds to “left”.

The endoscope system 11 includes the endoscope 15, the video processor 13, and a 3D monitor 17. The endoscope 15 is, for example, a medical flexible endoscope. The video processor 13 performs predetermined image processing on a captured image (for example, a still image or a moving image) captured by the endoscope 15 inserted into an observation target (for example, a blood vessel, a skin, an organ wall inside a human body, or the like) in the subject, and outputs the image to the 3D monitor 17. The 3D monitor 17 inputs a captured image (for example, a composite image in which a fluorescent portion in a fluorescent image that fluoresces in the IR (Infrared Ray) band is superimposed on a corresponding portion (that is, a coordinate in an image) in an Red Green Blue (RGB) image on an RGB image to be described later) output from the video processor 13 and having a left-right parallax after image processing, and displays the captured image in a stereoscopic manner (3D). In addition, when the simulmethod is used in the endoscope system 11, the 3D monitor 17 can input the captured image for the left eye and the captured image for the right eye output from the video processor 13, and can display the captured images in a stereoscopic manner (3D) after forming the left-right parallax. When only one of the composite images (see above) after the image processing is input from the video processor 13, the 3D monitor 17 may display the composite image in 2D. Examples of the image processing include, but are not limited to, color tone correction, gradation correction, and gain adjustment.

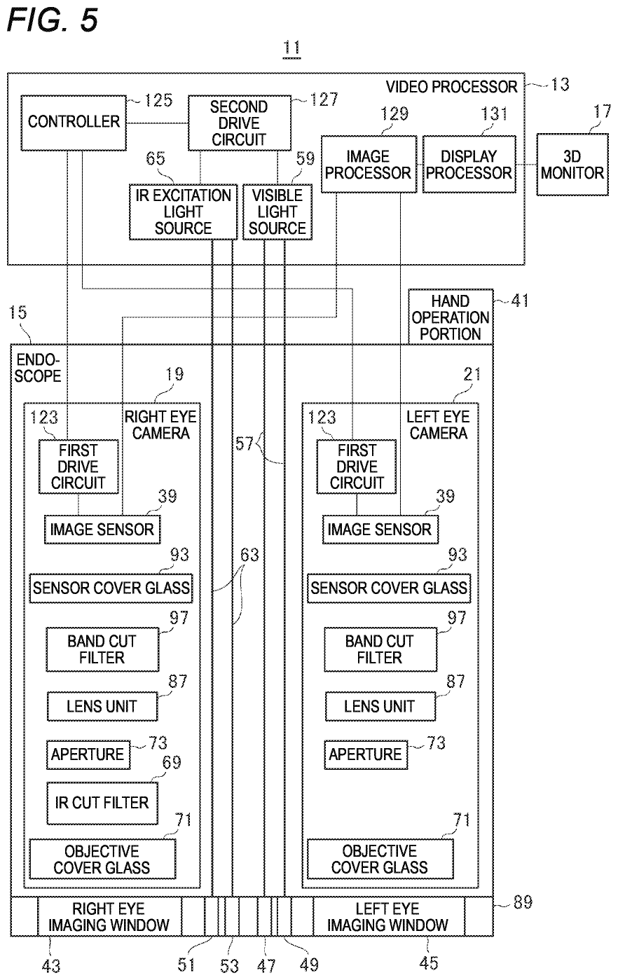

The endoscope 15 is inserted into a subject, which is, for example, a human body, and can capture an image of a 3D image of the observation target. The endoscope 15 includes a plurality of cameras, and each of the cameras captures a left eye image and a right eye image for composing the 3D image. Specifically, the endoscope 15 includes two cameras, that is, a right eye camera 19 (see FIG. 5) for capturing one image (for example, a right eye image) and a left eye camera 21 (see FIG. 5) for capturing the other image (for example, a left eye image) constituting the 3D image. In the first embodiment, a case where a plurality of cameras are two, the right eye camera 19 and the left eye camera 21, will be described, but the number of cameras is not limited thereto, and may be two or more, for example, three, four, or the like.

The endoscope 15 includes a scope 23 constituting an insertion distal end portion and inserted into an observation target, and a plug portion 25 to which a rear end portion of the scope 23 is connected. The scope 23 includes a flexible portion 27 having relatively long flexibility and a rigid portion 29 having rigidity provided at the distal end of the flexible portion 27. The structure of the scope 23 will be described later.

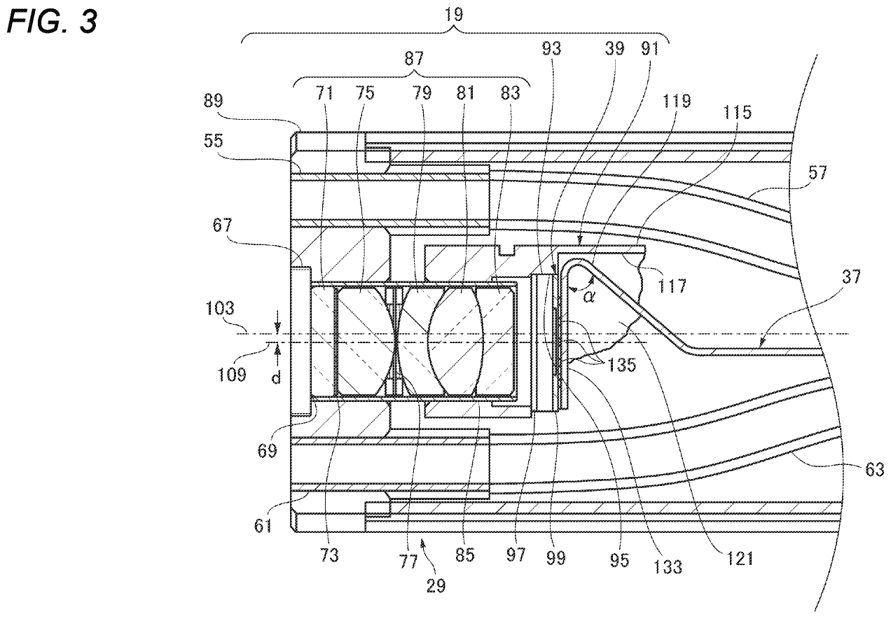

The video processor 13 includes a housing 31, performs image processing on the image captured by the endoscope 15, and outputs the image after the image processing to the 3D monitor 17 as display data. A socket portion 35 into which a proximal end portion 33 of the plug portion 25 is inserted is disposed on the front surface of the housing 31. Since the proximal end portion 33 of the plug portion 25 is inserted into the socket portion 35, and the endoscope 15 and the video processor 13 are electrically connected, electric power and various data or information (for example, captured image data or various control information) can be transmitted and received between the endoscope 15 and the video processor 13. These electric power and various data or information are transmitted from the plug portion 25 to the flexible portion side via a transmission cable 37 (see FIG. 3) inserted into the scope 23. Data of a captured image output from an image sensor 39 (see FIG. 3) provided inside the rigid portion 29 is transmitted from the plug portion 25 to the video processor 13 via the transmission cable 37. The flexible portion 27 is movable (for example, bent) in response to an input operation to a hand operation portion 41 (see FIG. 5) of the endoscope 15. The hand operation portion 41 of the endoscope 15 is disposed, for example, on the proximal end side of the endoscope 15 close to the video processor 13.

The video processor 13 performs predetermined image processing (see above) on the image data transmitted via the transmission cable 37, generates and converts the image data after the image processing as display data, and outputs the display data to the 3D monitor 17.

The 3D monitor 17 is configured using, for example, a display device such as a liquid crystal display (LCD), a cathode ray tube (CRT), or an organic electroluminescence (EL). The 3D monitor 17 displays the data of the image (that is, the image of the observation target captured by the endoscope 15) after the image processing is performed by the video processor 13. The image displayed on the 3D monitor 17 is visually recognized, for example, by a doctor or the like during surgery using the endoscope 15. In the first embodiment, as described above, the 3D monitor 17 can display the captured image of the observation target as a 3D image.

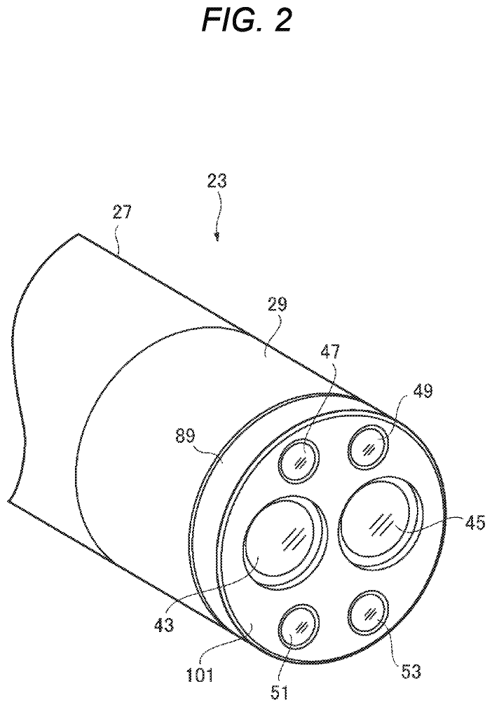

FIG. 2 is a perspective view showing the appearance of the distal end of the scope 23. A right eye imaging window 43, a left eye imaging window 45, a right eye white light irradiation window 47, a left eye white light irradiation window 49, a right eye excitation light irradiation window 51, and a left eye excitation light irradiation window 53 are disposed at the distal end of the scope 23. The right eye white light irradiation window 47 and the left eye white light irradiation window 49 and the right eye excitation light irradiation window 51 and the left eye excitation light irradiation window 53 may be disposed so as to be vertically interchanged, respectively.

The right eye white light irradiation window 47 and the left eye white light irradiation window 49 are disposed such that a white light illumination portion 55 (see FIG. 3) for irradiating the observation target with white light (that is, visible light of normal Red Green Blue (RGB)) is in contact with the right eye white light irradiation window and the left eye white light irradiation window, respectively. White light irradiated from a visible light source 59 (see FIG. 5) on the proximal end side is guided to the white light illumination portion 55 by an optical fiber 57 (see FIG. 3). Note that the visible light source 59 may not be disposed on the proximal end side, and, for example, a white LED (not shown) that can irradiate the white light illumination portion 55 with white light may be directly disposed. In this case, the white light irradiated from each white LED is irradiated from the right eye white light irradiation window 47 and the left eye white light irradiation window 49 to the observation target via the white light illumination portion 55.

An excitation light illumination portion 61 (see FIG. 3) for irradiating the observation target with excitation light (hereinafter referred to as “IR excitation light”) of the IR band (that is, the IR region) is in contact with the right eye excitation light irradiation window 51 and the left eye excitation light irradiation window 53, respectively. The excitation light illumination portion 61 guides the IR excitation light irradiated from an IR excitation light source 65 (see FIG. 5) on the proximal end side by an optical fiber 63 (see FIG. 3). Note that the IR excitation light source 65 is not disposed on the proximal end side, and for example, an IR-LED (not shown) capable of irradiating the excitation light illumination portion 61 with the excitation light of the IR band may be directly disposed. In this case, the excitation light of the IR band irradiated from each IR-LED is irradiated from the right eye excitation light irradiation window 51 and the left eye excitation light irradiation window 53 to the observation target via the excitation light illumination portion 61.

Here, the IR excitation light has a role of exciting a fluorescent agent (an aspect of a fluorescent substance) such as indocyanine green (ICG) having a property of being administered to a subject as a human body and accumulated in an affected part by irradiating a fluorescent agent (one aspect of the fluorescent substance) to emit fluorescence. The IR excitation light is near-infrared light having a wavelength band of, for example, about 690 nm to 820 nm. A right eye camera 19 (see FIG. 5) for capturing an image of an observation target is disposed on the back side (that is, the back surface side) of the right eye imaging window 43. Similarly, a left eye camera 21 (see FIG. 5) for imaging an observation target is disposed on the back side (that is, the back surface side) of the left eye imaging window 45.

In the first embodiment, the white light and the IR excitation light are exemplified as the types of the light to be irradiated, but the white light and the other special light may be irradiated. As the other special light, for example, excitation light in an ultraviolet region for exciting a fluorescent agent (an embodiment of a fluorescent substance) such as 5-ALA (aminolevulinic acid) may be used.

FIG. 3 is a longitudinal cross sectional view showing the configuration of the right eye camera 19 disposed in the scope. In the right eye camera 19, a negative lens 67, an IR cut filter 69, an objective cover glass 71, an aperture 73, a first lens 75, a spacer 77, a second lens 79, a third lens 81, and a fourth lens 83 are disposed along the optical axis in order from the observation target side (that is, the objective side). The IR cut filter 69 cuts (blocks) transmission of light in an IR band (that is, a wavelength band having a wavelength of 700 nm or more) incident on the right eye camera 19. That is, the IR cut filter 69 does not form an IR band light on the image sensor 39 disposed on the rear stage side (that is, the rear side) of the fourth lens 83, and forms an image of white light (that is, visible light) in a wavelength band less than 700 nm on the image sensor 39.

The IR cut filter 69, the objective cover glass 71, the aperture 73, the first lens 75, the spacer 77, the second lens 79, the third lens 81, and the fourth lens 83 are accommodated inside a tubular lens holder 85 to constitute a lens unit 87 which is an optical system. The lens unit 87 is held in a holding hole of a distal end flange portion 89 provided on the distal end of the rigid portion 29. The distal end flange portion 89 is formed in a substantially disc shape by a metal such as stainless steel, for example. In addition to the lens units 87 of the right eye camera 19 and the left eye camera 21, the distal end flange portion 89 also holds the white light illumination portion 55 and the excitation light illumination portion 61 in the holding holes, respectively.

A tubular imaging element holding member 91 is fixed to the outer periphery of the lens holder 85 on the rear portion of the lens unit 87 whose front portion is held by the distal end flange portion 89. The imaging element holding member 91 is made of a metal such as stainless steel. A sensor holding portion 95 for holding a sensor cover glass 93 and the image sensor 39 is formed on the inner periphery of the rear portion of the imaging element holding member 91. The imaging element holding member 91 positions and fixes the lens unit 87 and the image sensor 39 by fixing the image sensor 39 to the sensor holding portion 95. A band cut filter 97 is provided on the front surface of the sensor cover glass 93.

The band cut filter 97 cuts (blocks) transmission of light having a wavelength of 700 nm to 830 nm including a wavelength band of IR excitation light for exciting a fluorescent agent such as indocyanine green (ICG) administered to a subject to emit fluorescence, for example. The band cut filter 97 is formed on the front surface of the sensor cover glass 93 by, for example, vapor deposition.

The lens unit 87 collects light from the observation target (for example, white light reflected by an affected part or the like, fluorescence generated by fluorescent light emission of fluorescent agents of the affected part or the like), and forms an image on the imaging surface of the image sensor 39. The spacer 77 is disposed between the first lens 75 and the second lens 79, and stabilizes the positions of the first lens and the second lens. The objective cover glass 71 protects the lens unit 87 from the outside.

The imaging element holding member 91 positions and fixes the lens unit 87 and the sensor cover glass 93. The sensor cover glass 93 is disposed on the imaging surface of the image sensor 39, and protects the imaging surface. The image sensor 39 is, for example, a single plate type solid-state imaging element capable of simultaneously receiving IR light, red light, blue light, and green light. The image sensor 39 has a sensor substrate 99 on the back surface.

The left eye camera 21 has the same configuration as the right eye camera 19 shown in FIG. 3 except that the left eye camera does not include the IR cut filter 69 shown in FIG. 3. That is, in the left eye camera 21, the negative lens 67, the objective cover glass 71, the aperture 73, the first lens 75, the spacer 77, the second lens 79, the third lens 81, and the fourth lens 83 are accommodated in the lens holder 85 along the optical axis from the observation target side (that is, the objective side), and constitute the lens unit 87. The front portion of the lens unit 87 is fixed to the distal end flange portion 89, and the sensor holding portion 95 is fixed to the rear portion of the lens unit. The sensor holding portion 95 holds the sensor cover glass 93 and the image sensor 39. The band cut filter 97 is provided on the front surface of the sensor cover glass 93.

In FIG. 3, the band cut filter 97 is formed on the front surface of the sensor cover glass 93, but may be formed on the back surface of the objective cover glass 71.

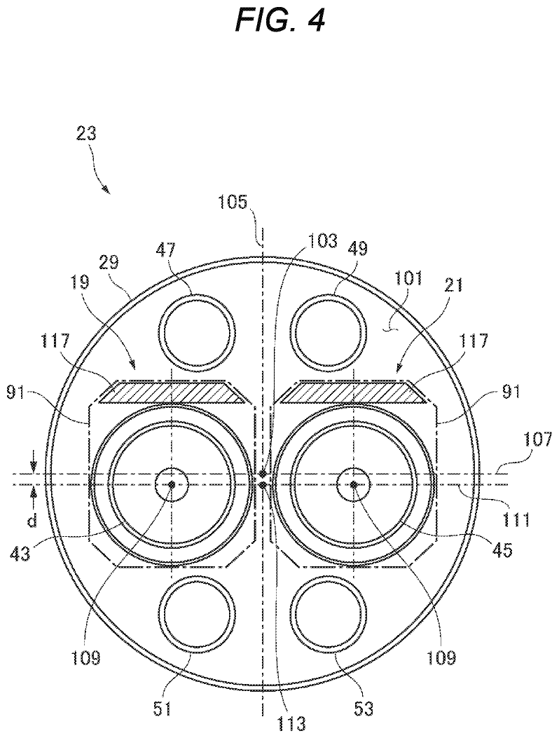

FIG. 4 is a front view of the rigid portion 29 as viewed from the subject side. In the endoscope 15, the distal end surface 101 of the rigid portion 29 has a substantially circular shape. Here, a first virtual line 105 orthogonal to an axis line 103 (see FIG. 3) of the rigid portion 29 is set on the distal end surface 101 of the rigid portion 29. In addition, a second virtual line 107 orthogonal to the axis line 103 and the first virtual line 105 is set on the distal end surface 101 of the rigid portion 29. In this case, in the endoscope 15, the plurality of cameras (the right eye camera 19 and the left eye camera 21) are disposed on the left and right sides of the rigid portion 29 sandwiching the first virtual line 105.

The plurality of cameras (the right eye camera 19 and the left eye camera 21) are disposed such that each imaging center 109 is shifted in a direction along the first virtual line 105 with respect to the second virtual line 107. The imaging center 109 is one point on the imaging axis. The imaging center 109 may be the imaging axis itself. In the endoscope 15 according to the first embodiment, the right eye camera 19 and the left eye camera 21 are shifted to the lower side in FIG. 4.

Here, the imaging axis is an axis line passing through the center of the imaging surface (light receiving surface) of the image sensor 39 mounted on each of the right eye camera 19 and the left eye camera 21. The imaging center 109 may be the center of the imaging surface (light receiving surface) of the image sensor 39 mounted on each of the right eye camera 19 and the left eye camera 21, or may be an axis line passing through the center thereof. For example, when both the lens unit 87 and the image sensor 39 in each of the right eye camera 19 and the left eye camera 21 are disposed parallel to the axis 103 (see FIG. 3), the imaging axis passes through the imaging center 109. Note that the imaging center 109 may or may not be aligned with the center (that is, the imaging window center) of the negative lens 67 of the distal end surface 101.

In the endoscope 15 according to the first embodiment, two camera, the right eye camera 19 and the left eye camera 21, are disposed such that a line segment 111 connecting the centers of respective imaging window is parallel to the second virtual line 107 on the distal end surface 101. Therefore, the midpoint 113 of the line segment 111 is disposed so as to be separated (offset) from the axis line 103 by a distance d.

In the endoscope 15, each of the right eye camera 19 and the left eye camera 21 includes a lens unit 87 that is coaxial with the imaging center 109. In the imaging element holding member 91 shown in FIG. 3, the image sensor 39 is positioned and fixed to the lens unit 87.

As illustrated in FIG. 3, the imaging element holding member 91 includes a cable accommodating portion 115 that accommodates a transmission cable 37 connected to the image sensor 39. A flexible substrate can be used as the transmission cable 37.

As the flexible substrate, for example, an flexible flat cable (FFC) in which a conductor formed of a plurality of band-shaped thin plates is covered with an insulating sheet material and which is formed in a flexible band-shaped cable, or an flexible printed wiring board (FPC) in which a linear conductor is pattern-printed on a flexible insulating substrate can be used.

Each of the flexible substrates conductively connected to each of the right eye camera 19 and the left eye camera 21 is accommodated in the cable accommodating portion 115. In the flexible substrate, each of the circuit conductors is integrated with the transmission cable 37, and the flexible substrate is inserted into the scope 23.

The cable accommodating portion 115 includes a protruding portion 117. The protruding portion 117 accommodates a bent portion 119 of the transmission cable 37 protruding from the outer shape of the image sensor 39 in a direction orthogonal to the axis line 103. The protruding portion 117 is formed by thinning a wall portion of the cable accommodating portion 115 formed in a cylindrical shape on the imaging element holding member 91. The imaging element holding member 91 protrudes outward from the imaging center 109 in the radial direction on a side where the protruding portion 117 is located. In the first embodiment, as shown in FIG. 4, the protruding portion 117 protrudes upward from the upper side of each of the imaging element holding members 91 of the right eye camera 19 and the left eye camera 21.

In the endoscope 15 according to the first embodiment, the imaging center 109 coaxial with the lens units 87 of the right eye camera 19 and the left eye camera 21 is disposed to be shifted in the direction (downward direction) along the first virtual line 105 in the direction opposite to the protruding direction (upward direction) of the protruding portion 117.

The bent portion 119 accommodated in the protruding portion 117 is bent at an acute angle a. Since the transmission cable 37 is disposed in the vicinity of the axis line 103 by being bent at an acute angle a immediately after being connected to the image sensor 39, and can be inserted along the vicinity of the axis line of the scope 23.

The protruding portion 117 is filled with an adhesive material 121. The bent portion 119 accommodated in the protruding portion 117 is embedded in the adhesive material 121, thereby being fixed and held integrally with the imaging element holding member 91.

Next, a hardware configuration example of the endoscope system 11 according to the first embodiment will be described.

FIG. 5 is a block diagram showing a hardware configuration example of the endoscope system 11 according to the first embodiment. The endoscope 15 includes a right eye camera 19 and a left eye camera 21 provided in the rigid portion 29, and a first drive circuit 123.

The first drive circuit 123 operates as a drive portion to switch on and off the electronic shutter of the image sensor 39. Note that the first drive circuit 123 is not disposed in either the right eye camera 19 or the left eye camera 21, and may be disposed in the video processor 13. When the electronic shutter is turned on by the first drive circuit 123, the image sensor 39 photoelectrically converts the optical image formed on the imaging surface and outputs an image signal. In the photoelectric conversion, exposure of an optical image and generation and reading of an image signal are performed.

The IR cut filter 69 is disposed on the light receiving side of the image sensor 39, blocks the IR excitation light reflected by the subject among the light passing through the lens, and transmits the light of fluorescent light emission and visible light excited by IR excitation light. In FIG. 5, the IR cut filter 69 is provided on only one of them (for example, the right eye camera 19), but the IR cut filter 69 may be provided on both (that is, the right eye camera 19 and the left eye camera 21) or may not be provided on both.

The video processor 13 includes a controller 125, a second drive circuit 127, an IR excitation light source 65, a visible light source 59, an image processor 129, and a display processor 131.

The controller 125 includes a processor configured using, for example, a central processing unit (CPU), a digital signal processor (DSP), or a field programmable gate array (FPGA), and performs overall control of execution of various operations related to imaging processing by the endoscope 15 by the processor. The controller 125 controls the presence/absence of light emission to the second drive circuit 127. In addition, the controller 125 executes drive control for switching on and off of the electronic shutter with respect to the first drive circuit 123 provided in each of the right eye camera 19 and the left eye camera 21.

The second drive circuit 127 is, for example, a light source drive circuit, and drives the IR excitation light source 65 under the control of the controller 125 to continuously emit the IR excitation light. The IR excitation light source 65 continuously lights up in the imaging period, and continuously irradiates the subject with the IR excitation light.

This imaging period indicates a period during which the observation region is imaged by the endoscope 15. The imaging period is, for example, a period from when the endoscope system 11 receives a user operation to turn on a switch provided in the video processor 13 or the endoscope 15 until receiving a user operation to turn off the switch.

The second drive circuit 127 may drive the IR excitation light source 65 to emit the IR excitation light at a predetermined interval. In this case, the IR excitation light source 65 performs intermittent lighting (pulse lighting) in the imaging period, and pulse-irradiates the subject with the IR excitation light. In the imaging period, the timing at which the IR excitation light is emitted and the visible light is not emitted is the timing at which the fluorescent light emission image is captured.

The IR excitation light source 65 has a laser diode (LD, not shown), and emits a laser light (an example of IR excitation light) having a wavelength in the wavelength band of 690 nm to 820 nm guided by the optical fiber 57 from the LD. Since the mode of the fluorescent light emission changes according to the concentration of the chemical such as the ICG or the physical condition of the patient as the subject, a plurality of (for example, 780 nm, 808 nm) of the laser lights having the wavelength in the wavelength band of 690 nm to 820 nm may be emitted at the same time.

The second drive circuit 127 drives the visible light source 59 to pulse-emit visible light (for example, white light). The visible light source 59 pulse-irradiates the subject with visible light at the timing of imaging the visible light image in the imaging period. In general, the light of fluorescent light emission has a weak brightness. On the other hand, visible light can obtain strong light even with a short pulse.

The light source device of the endoscope system 11 alternately outputs visible light and excitation light. In the endoscope system 11, the irradiation timing of the visible light and the imaging timing of the fluorescence image generated by the excitation light do not overlap each other.

The image processor 129 performs image processing on the fluorescence emission image and the visible light image alternately output from the image sensor 39, and outputs the image data after the image processing.

For example, when the luminance of the fluorescence emission image is lower than the luminance of the visible light image, the image processor 129 adjusts the gain as a gain controller so as to increase the gain of the fluorescence emission image. Instead of increasing the gain of the fluorescence emission image, the image processor 129 may adjust the gain by decreasing the gain of the visible light image. The image processor 129 may adjust the gain by increasing the gain of the fluorescence emission image and decreasing the gain of the visible light image. The image processor 129 may adjust the gain by increasing the gain of the fluorescence emission image to be larger than that of the visible light image and increasing the gain of the visible light image.

The display processor 131 converts the image data output from the image processor 129 into a display signal such as a national television system committee (NTSC) signal suitable for video display, and outputs the display signal to the 3D monitor 17.

The 3D monitor 17 displays the fluorescence emission image and the visible light image in the same area, for example, in accordance with the display signal output from the display processor 131. The 3D monitor 17 displays the visible light image and the fluorescence image on the same screen in a superimposed manner or individually. As a result, the user can check the observation target with high accuracy while superimposing the fluorescence emission image and the visible light image displayed on the 3D monitor 17 on the same captured image or individually viewing the same.

Next, in addition to the image processing for displaying the fluorescence emission image and the visible light image described above, the outline of the extension function enabling the endoscope system 11 by the compound eye of the endoscope 15 will be described.

### Wavelength Characteristic Enhancement Processing

In the endoscope system 11, the video processor 13 (for example, the image processor 129) as an example of a processor may perform image processing on a plurality of captured images having different wavelength characteristics captured by each of the pair of the right eye camera 19 and the left eye camera 21 which are interlocked with each other, generate a composite image by extracting differences between the respective captured images, and display the composite image on the 3D monitor 17. In this case, each of the right eye camera 19 and the left eye camera 21 may be a camera having the same specification. The right eye camera 19 and the left eye camera 21 may be cameras having different specifications. In this case, the visible light camera can omit the IR cut filter 69.

### Distance Measurement Processing

Further, in the endoscope system 11, the video processor 13 (for example, the image processor 129) as an example of the processor can measure the distance from the endoscope 15 to the subject due to the parallax appearing in the pair of captured images captured by each of the pair of interlocked cameras. In this case, each of the right eye camera 19 and the left eye camera 21 is a camera having the same specification. Here, the parallax means that the appearance of the object mutual position in the space changes depending on the observation position. The distance to the subject can be measured by triangulation using, for example, a distance (known) between two cameras.

As a condition in this case, two cameras completely match the specifications. The optical axes of the two cameras are parallel to each other. Only the optical axes of the two cameras are separated by a certain distance. Under these imaging conditions, the imaging surfaces of the two cameras are set to be the same plane. At this time, a plane passing through the same point in the two optical axes and the subject is an epipolar plane. An intersection line between the epipolar plane and the image plane is an epipolar line.

Under the conditions described above, the parallax is the difference in the coordinates of the images at the same point on the same subject on the two captured images. When obtaining the parallax, the two cameras are placed in parallel or the like, so that the search for the same point is done in one dimension in the linear direction. This is because there is a condition of epipolar constraint between the captured images of the two cameras. The epipolar constraint is a phenomenon in which a point shown in one camera is projected onto the epipolar line of the other camera. The distance to the subject can be obtained by the positional deviation (that is, the parallax) of the same point in the captured images captured by the two cameras in this manner. Since a specific formula for calculating the distance to the subject by parallax is well known, the description thereof is omitted here.

### Stereoscopic Image Processing

In addition, in the endoscope system 11, the video processor 13 (for example, the image processor 129) as an example of a processor can perform image processing on each of a pair of captured images on which parallax is formed, which are captured by each of a pair of interlocked cameras, generate a stereoscopic image reflecting the depth information, and display the stereoscopic image on the 3D monitor 17. In this case, each of the right eye camera 19 and the left eye camera 21 is a camera having the same specification.

### Depth of Field Enhancement Processing

In addition, in the endoscope system 11, the video processor 13 (for example, the image processor 129) as an example of a processor can perform image processing on a pair of captured images having different focal lengths captured by a pair of interlocked cameras, generate a composite image with a deep depth of field (depth synthetic image), and display the composite image on the 3D monitor 17. In this case, the right eye camera 19 and the left eye camera 21 are cameras having different specifications.

### Hybrid Zoom Processing

In addition, in the endoscope system 11, the video processor 13 (for example, the image processor 129) as an example of a processor can perform image processing on a plurality of captured images of different angles of view or magnifications captured by a pair of interlocked cameras, generate a composite image in which different fields of view are simultaneously captured, and display the composite image on the 3D monitor 17. A so-called bird's eye camera (simultaneous zooming and panning) can be performed. In this case, the right eye camera 19 and the left eye camera 21 are cameras having different specifications.

### High Sensitivity Processing

In the endoscope system 11, a monochrome image sensor 39 may be provided in one camera, and a color image sensor 39 may be provided in the other camera. Generally, the monochrome image sensor 39 has a higher ISO sensitivity than the color image sensor 39. In the endoscope system 11, a portion that cannot be imaged due to insufficient light intensity by the color image sensor 39 can be complemented by image information captured by the monochrome image sensor 39, and a finer composite image can be obtained.

Next, the function of the endoscope 15 according to the first embodiment will be described.

The endoscope 15 according to the first embodiment includes a rigid portion 29 provided at the distal end of the scope 23, which is formed in a substantially cylindrical shape (including a circular shape. The same applies below.) and whose distal end surface 101 has a substantially circular shape. The endoscope 15 includes a plurality of cameras disposed on the left and right sides of the rigid portion 29 sandwiching the first virtual line 105 orthogonal to the axis line 103 of the rigid portion 29 on the distal end surface 101. The plurality of cameras include a first camera (for example, a right eye camera 19 or a left eye camera 21), and the first camera is disposed such that an imaging center 109 is shifted from a second virtual line 107 orthogonal to each of the axis line 103 and the first virtual line 105 in a direction along the first virtual line 105.

In the endoscope 15 according to the first embodiment, a plurality of cameras (for example, the right eye camera 19 and the left eye camera 21) are disposed inside the rigid portion 29 formed in a cylindrical shape. The right eye camera 19 and the left eye camera 21 take in the imaging light from the imaging window and obtain the information of the subject as image data. Here, it is assumed that there is only one camera, and the projection shape in the direction along the imaging center 109 of the camera is substantially circular. In addition, it is assumed that the imaging window which is integrated with the camera needs to have the largest occupied area on the distal end surface 101 of the rigid portion 29 as compared with other members. Further, it is assumed that a plurality of other members (for example, light irradiation windows) are disposed in point symmetry on the distal end surface 101. In this case, the camera is provided such that the imaging center 109 is aligned with the axis line 103 of the rigid portion 29, it is easy to avoid interference with other members disposed around the camera.

On the other hand, a case where there are a plurality of cameras is considered. Here, a first virtual line 105 orthogonal to the axis line 103 of the rigid portion 29 is set on the distal end surface 101 of the rigid portion 29. In addition, a second virtual line 107 orthogonal to the axis line 103 and the first virtual line 105 is set on the distal end surface 101 of the rigid portion 29. That is, the first virtual line 105 and the second virtual line 107 are orthogonal to each other in an XY coordinate axis shape at the axial position of the distal end surface 101. In a case where the plurality of other members are disposed above and below the second virtual line 107, the plurality of cameras (the right eye camera 19 and the left eye camera 21) can be disposed side by side in the direction along the second virtual line 107 between the other members, so that it is possible to easily avoid interference with other members. That is, the right eye camera 19 and the left eye camera 21 are disposed such that the imaging center 109 is on the second virtual line.

However, in some cases, the outer shape of the camera is not limited to a substantially circular shape. That is, the camera has the shape such that a part protrudes from a substantially circular shape centered on the imaging center 109. In this case, in the camera, when the imaging center 109 is disposed on the second virtual line and the protruding portion 117 is in any one of the directions along the first virtual line 105 (one of the upper/lower directions in the above example), the protruding portion 117 easily interferes with other members in the housing of the endoscope 15.

On the other hand, by disposing the protruding portion 117 on both the left and right sides in the direction along the second virtual line 107, there is also a disposition in which the disposition space of the protruding portion 117 is twisted (disposition in which the space of the side portion of the camera is used). In this method, for example, when there are two cameras, it is necessary to prepare a transmission cable 37 having a different shape for each of the left and right cameras. When each of the transmission cables 37 of different types is aligned for each of the plurality of cameras, the component management becomes complicated, and the component cost increases.

Therefore, in the endoscope 15 according to the first embodiment, the right eye camera 19 and the left eye camera 21 disposed on the left and right of the rigid portion 29 are disposed such that the imaging center 109 is shifted in the direction along the first virtual line 105 with respect to the second virtual line 107. The amount of deviation is, for example, half of the protruding dimension of the protruding portion 117. Thereby, in a limited space surrounded by the other members inside the circular shape, the disposition area of the other members in the housing of the endoscope 15 can also be secured (the light irradiation window can also be secured to the maximum).

That is, the center of the maximum outer diameter of the camera is aligned with the center of the free space sandwiched between the upper and lower other members, so that a layout without wasting free space is possible. In this case, the imaging center 109 is displaced from the second virtual line 107 passing through the axis line 103 of the rigid portion 29. The imaging center 109 is separated from the axis line 103, but according to such an offset arrangement, it is possible to realize a high density component disposition in a limited accommodation space of the rigid portion 29, and it is possible to obtain a large effect of suppressing an increase in the outer diameter of the rigid portion 29. In this case, the transmission cable 37 does not have left-right hand movement. As a result, in the rigid portion 29, the endoscope 15 is disposed such that the right eye camera 19 and the left eye camera 21 are offset as described above, so that it is possible to suppress an increase in the outer diameter of the rigid portion 29 while using the transmission cable 37 as a common component.

Therefore, according to the endoscope 15 according to the first embodiment, it is possible to suppress an increase in the outer diameter in the endoscope 15 with two or more eyes.

In the endoscope 15, the plurality of cameras include the second camera (for example, the left eye camera 21 or the right eye camera 19), and the first camera (for example, the right eye camera 19 or the left eye camera 21) and the second camera (for example, the left eye camera 21 or the right eye camera 19) are disposed such that the line segment 111 connecting the centers of the respective imaging windows on the distal end surface 101 is parallel to the second virtual line 107, and the midpoint 113 of the line segment 111 is separated from the axis line 103.

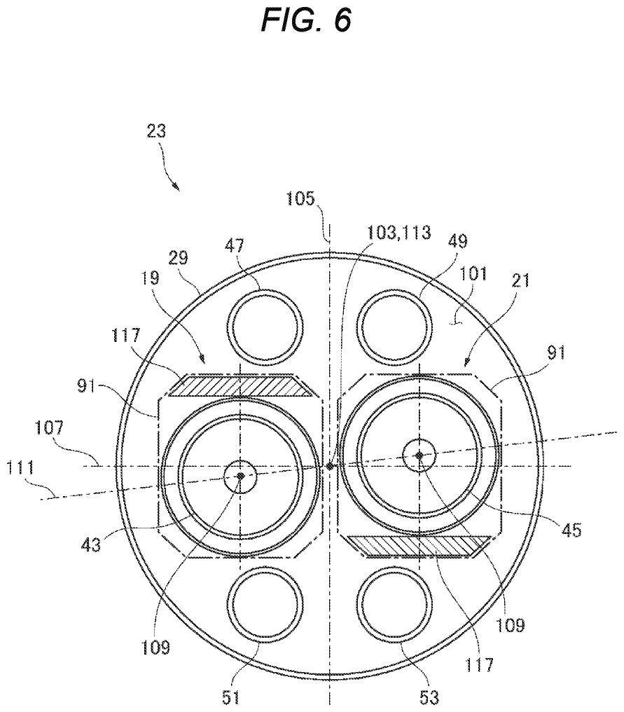

In the endoscope 15, the line segment 111 connecting the centers of the imaging windows of the right eye camera 19 and the left eye camera 21 is parallel to the second virtual line 107. A midpoint 113 of the line segment 111 is separated from the axis line 103. The direction in which the line segment 111 is separated is opposite to the protruding portion 117. That is, the imaging center 109 positioned on the line segment 111 is disposed to move to the side opposite to the protruding portion 117 with respect to the second virtual line 107. In this configuration, since the line segment 111 connecting the two imaging centers 109 is parallel to the second virtual line 107, the layout (see, for example, FIG. 6) in which the two cameras are disposed to be vertically shifted with respect to the second virtual line 107 is eliminated.

Note that, when the two cameras are disposed so as to be vertically shifted from each other with the second virtual line 107 interposed therebetween (that is, in the case of FIG. 6), it is necessary to reverse the upper and lower sides of the acquired images of the respective cameras. That is, the protruding portion 117 of one of the cameras is on the upper side, and the protruding portion 117 of the other camera is on the lower side. In this case, image processing for rotating one image data by 180° is required.

On the other hand, in the endoscope 15 in which the right eye camera 19 and the left eye camera 21 are disposed such that the line segment 111 connecting the two imaging centers 109 is parallel to the second virtual line 107, since the vertical imaging direction of the subject is not reversed, unnecessary image processing can be omitted. As a result, since the endoscope 15 offsets the right eye camera 19 and the left eye camera 21 in the same direction (lower direction in the example described above) with respect to the imaging centers 109 in order to eliminate unnecessary space, it is possible to suppress the increase in the outer diameter of the rigid portion 29 and prevent complicated image processing from occurring while the transmission cable 37 is used as a common component.

In the endoscope 15, the first camera (for example, the right eye camera 19 or the left eye camera 21) includes a lens unit 87 that is coaxial with the imaging center 109, and an imaging element holding member 91 that positions and fixes the image sensor 39 to the lens unit 87.

In the endoscope 15, the lens unit 87 is disposed coaxially with the imaging center 109. In this case, the distal end side of each lens unit 87 is fixed and supported by a metallic distal end flange portion 89 provided at the distal end of the rigid portion 29. The imaging element holding member 91 is fixed by inserting the inner periphery of the holding hole into the outer periphery of the rear end side of each lens unit 87. An image sensor 39 in which a light receiving surface is disposed on the imaging side of the lens unit 87 is fixed to the imaging element holding member 91 fixed to the lens unit 87. That is, the imaging element holding member 91 fixes and holds the image sensor 39 while positioning the lens unit 87 and the image sensor 39 in each of the right eye camera 19 and the left eye camera 21. Accordingly, the right eye camera 19 and the left eye camera 21 can integrally position and fix the lens units 87 and the image sensors 39 to the high strength distal end flange portion 89 while suppressing the increase of the outer diameter of the rigid portion 29 with a simple structure (small number of components).

Further, in the endoscope 15, the imaging element holding member 91 includes a cable accommodating portion 115 which accommodates the transmission cable 37 connected to the image sensor 39.

In the endoscope 15, the transmission cable 37 for extracting the electric signal from the image sensor 39 can be secured in the imaging element holding member 91 in the limited inner space of the rigid portion 29. Since the transmission cable 37 is passed from the rigid portion 29 of the scope 23 to the plug portion 25, an external stress acts on the transmission cable due to the bending of the flexible portion 27. In the transmission cable 37, the cable conductor in the flexible substrate portion 133 at the distal end is conductively connected to a plurality of bumps 135 (see FIG. 3) provided on the sensor substrate 99 by soldering or the like. The external stress acting on the transmission cable 37 may adversely affect the fine cable connection portion (soldered portion with the bumps 135) between the transmission cable 37 and of the image sensor 39. Therefore, by providing the cable accommodating portion 115 that can fix the end portion of the transmission cable 37 together with the image sensor 39 to the image sensor holding member 91 that fixes the image sensor 39, it is possible to block external stress that adversely affects the cable connection portion while suppressing the increase of the outer diameter of the rigid portion 29 without using another fixing member.

Further, in the endoscope 15, the cable accommodating portion 115 includes the protruding portion 117 that accommodates the bent portion 119 of the transmission cable 37 protruding from the outer shape of the image sensor 39 in the direction orthogonal to the axis line 103.

In the endoscope 15, the protruding portion 117 that accommodates the bent portion 119 of the transmission cable 37 is formed in the cable connection portion of the imaging element holding member 91. In the image sensor 39, a large number of bumps 135 for connection to the transmission cable 37 are vertically and horizontally disposed on the back surface of the quadrangular sensor substrate 99. When the number of the bumps 135 increases with an increase in the amount of information, the bumps are disposed with substantially the same area as the sensor substrate 99. In this case, the bumps 135 are connected to each other by a quadrangular flexible substrate disposed in parallel with the sensor substrate 99. The flexible substrate may be the same as or a part of the end of the transmission cable 37. Therefore, the flexible substrate of the transmission cable 37 formed with substantially the same area as the sensor substrate 99 needs to be bent at any one of the sides of the quadrangle to be used as the transmission cable 37. As a result, the bent portion of the transmission cable 37 becomes the bent portion 119 and protrudes from the outer shape of the sensor substrate 99 in the direction orthogonal to the axis line 103. The imaging element holding member 91 is provided with a protruding portion 117 that accommodates the bent portion 119 in the cable accommodating portion 115. By providing the protruding portion 117 in the cable accommodating portion 115, the imaging element holding member 91 can protect the bent portion 119 so as not to interfere with other members.

In the endoscope 15, the imaging center 109 is disposed to be shifted in the direction along the first virtual line 105 in the direction opposite to the protruding direction of the protruding portion 117.

In the endoscope 15, in the distal end surface 101 of the rigid portion 29, the protruding portion 117 accommodating the bent portion 119 protrudes beyond the outer shape of the imaging window. The protruding portion 117 serves as a necessary portion for protecting the bent portion 119. The protruding portion 117 of the imaging element holding member 91 protrudes from the outer shape. Therefore, in the endoscope 15, the right eye camera 19 and the left eye camera 21 are offset such that the imaging center 109 is disposed so as to be shifted in the direction opposite to the protruding direction of the protruding portion 117, so that a useless space is omitted.

In the endoscope 15, the bent portion 119 is bent at an acute angle.

In the endoscope 15, the bent portion 119 protruding beyond the outer shape of the imaging window can be returned to the vicinity of the center of the imaging window. Accordingly, in the endoscope 15, it is possible to suppress an increase in the outer diameter of the rigid portion 29 due to interference of the transmission cable 37 with other members.

In the endoscope 15, the bent portion 119 is embedded in the adhesive material 121 filled in the protruding portion 117.

In the endoscope 15, the bent portion 119 is embedded in the adhesive material 121 filled in the protruding portion 117, whereby the bent portion is integrally fixed to the imaging element holding member 91. As a result, the protruding portion 117 of the imaging element holding member 91 can reinforce and protect the bent portion 119 in which the internal stress has already been generated by bending so as not to cause any further displacement due to the external stress.

Next, a modification of the endoscope 15 according to the first embodiment will be described. In the following description, the same reference numerals as those of the endoscope 15 according to the first embodiment are used for the reference numerals used for the endoscope in each modification.

FIG. 6 is a front view of a first modification of the endoscope 15 according to the first embodiment. In the endoscope 15 according to the first modification, the protruding portion 117 of the right eye camera 19 is disposed on the upper side, and the protruding portion 117 of the left eye camera 21 is disposed on the lower side. In this case, the midpoint 113 of the line segment 111 connecting the two imaging centers 109 is aligned with the axis line 103, but each imaging center 109 is offset from the second virtual line 107.

According to the endoscope 15 according to the first modification, the same transmission cable 37 can be used. In this case, depending on the shape of the transmission cable 37, the layout may be advantageous.

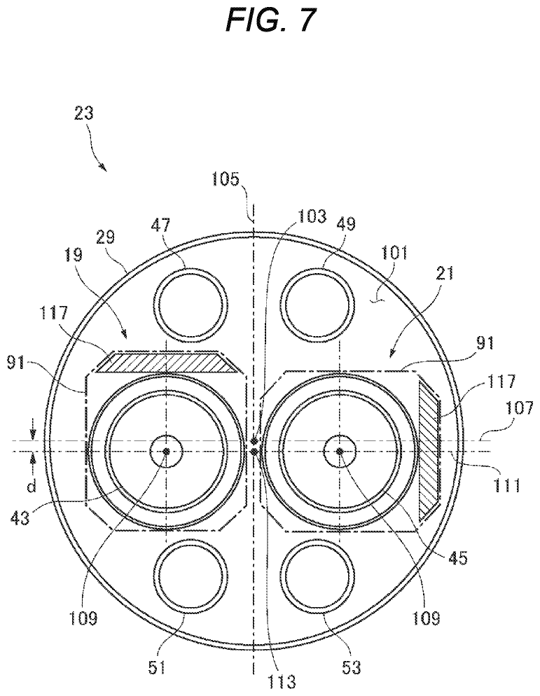

FIG. 7 is a front view of a second modification of the endoscope 15 according to the first embodiment. In the endoscope 15 according to the second modification, the protruding portion 117 of the right eye camera 19 is disposed on the upper side, and the protruding portion 117 of the left eye camera 21 is disposed on the left side.

According to the endoscope 15 of the second modification, two types of flexible substrates are provided, but the right eye camera 19 and the left eye camera 21 can be easily assembled to the rigid portion 29, respectively.

In the case of the second modification of FIG. 7, since the protruding portion 117 of the left eye camera 21 is disposed on the left side and the space between the left eye white light irradiation window 49 and the left eye excitation light irradiation window 53 is not used, the left eye camera 21 may not be disposed such that the imaging center 109 thereof is shifted downward along the first virtual line 105 with respect to the second virtual line 107. That is, the imaging center 109 of the left eye camera 21 may be disposed on the upper side with respect to the second virtual line 107, or may be disposed on the second virtual line 107.

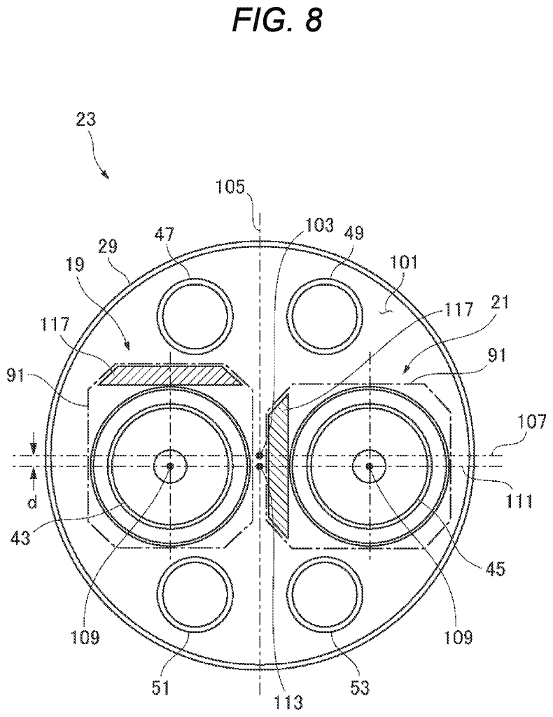

FIG. 8 is a front view of a third modification of the endoscope 15 according to the first embodiment. In the endoscope 15 according to the third modification, the protruding portion 117 of the right eye camera 19 is disposed on the upper side, and the protruding portion 117 of the left eye camera 21 is disposed on the right side.

According to the endoscope 15 of the third modification, two types of flexible substrates are provided, but the right eye camera 19 and the left eye camera 21 can be easily assembled to the rigid portion 29, respectively.

In the case of the third modification of FIG. 8, since the protruding portion 117 of the left eye camera 21 is disposed on the right side and the space between the left eye white light irradiation window 49 and the left eye excitation light irradiation window 53 is not used, the left eye camera 21 may not be disposed such that the imaging center 109 thereof is shifted downward along the first virtual line 105 with respect to the second virtual line 107. That is, the imaging center 109 of the left eye camera 21 may be disposed on the upper side with respect to the second virtual line 107, or may be disposed on the second virtual line 107.

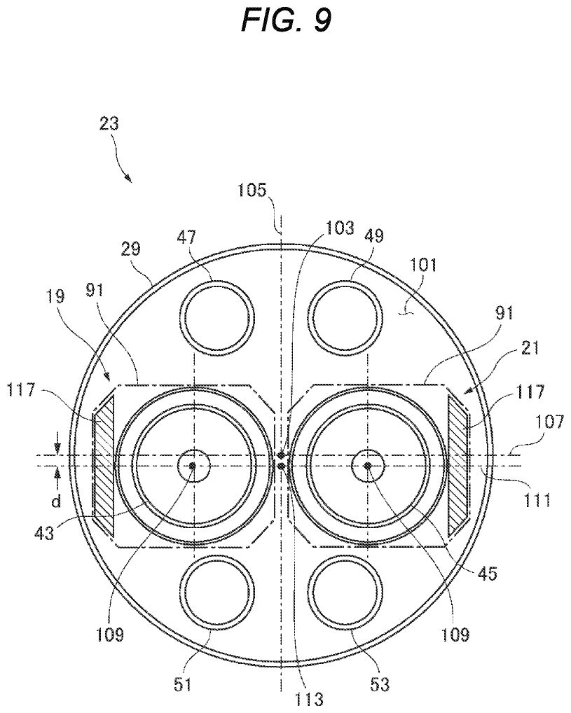

FIG. 9 is a front view of a fourth modification of the endoscope 15 according to the first embodiment. In the endoscope 15 according to the fourth modification, the protruding portion 117 of the right eye camera 19 is disposed on the right side, and the protruding portion 117 of the left eye camera 21 is disposed on the left side.

According to the endoscope 15 of the fourth modification, two types of flexible substrates are provided, but the right eye camera 19 and the left eye camera 21 can be easily assembled to the rigid portion 29, and the space in the upper-lower direction of the endoscope 15 can be maximized.

In the case of the fourth modification of FIG. 9, since the protruding portion 117 of the right eye camera 19 is disposed on the right side and the space between the right eye white light irradiation window 47 and the right eye excitation light irradiation window 51 is not used, the right eye camera 19 may not be displaced such that the imaging center 109 thereof is shifted downward along the first virtual line 105 with respect to the second virtual line 107. That is, the imaging center 109 of the right eye camera 19 may be disposed on the upper side with respect to the second virtual line 107, or may be disposed on the second virtual line 107. Similarly, since the protruding portion 117 of the left eye camera 21 is disposed on the left side and the space between the left eye white light irradiation window 49 and the left eye excitation light irradiation window 53 is not used, the left eye camera 21 may not be disposed such that the imaging center 109 thereof is shifted downward along the first virtual line 105 with respect to the second virtual line 107. That is, the imaging center 109 of the left eye camera 21 may be disposed on the upper side with respect to the second virtual line 107, or may be disposed on the second virtual line 107.

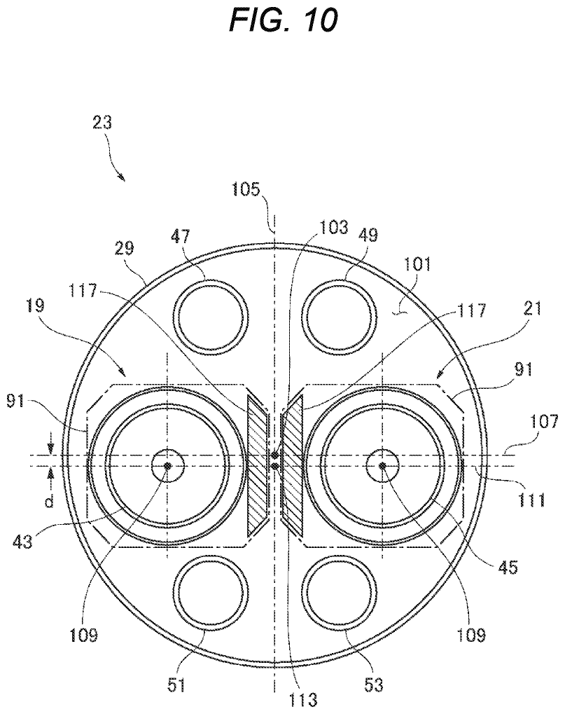

FIG. 10 is a front view of a fifth modification of the endoscope 15 according to the first embodiment. In the endoscope 15 according to the fifth modification, the protruding portion 117 of the right eye camera 19 is disposed on the left side, and the protruding portion 117 of the left eye camera 21 is disposed on the right side.

According to the endoscope 15 according to the fifth modification, two types of flexible substrates are provided, but the convergence angle when the 3D video is captured by the right eye camera 19 and the left eye camera 21 can be maximized.

In the case of the fifth modification of FIG. 10, since the protruding portion 117 of the right eye camera 19 is disposed on the left side and the space between the right eye white light irradiation window 47 and the right eye excitation light irradiation window 51 is not used, the right eye camera 19 may not be disposed such that the imaging center 109 thereof is shifted downward along the first virtual line 105 with respect to the second virtual line 107. That is, the imaging center 109 of the right eye camera 19 may be disposed on the upper side with respect to the second virtual line 107, or may be disposed on the second virtual line 107. Similarly, since the protruding portion 117 of the left eye camera 21 is disposed on the right side and the space between the left eye white light irradiation window 49 and the left eye excitation light irradiation window 53 is not used, the left eye camera 21 may not be disposed such that the imaging center 109 thereof is shifted downward along the first virtual line 105 with respect to the second virtual line 107. That is, the imaging center 109 of the left eye camera 21 may be disposed on the upper side with respect to the second virtual line 107, or may be disposed on the second virtual line 107.

FIG. 11 is a front view of a sixth modification of the endoscope 15 according to the first embodiment. In the endoscope 15 according to the sixth modification, the protruding portion 117 of the right eye camera 19 is disposed on the left side, and the protruding portion 117 of the left eye camera 21 is disposed on the left side.

According to the endoscope 15 according to the sixth modification, a flexible substrate having the same shape can be used, and the component cost during manufacturing can be reduced.

In the case of the sixth modification of FIG. 11, since the protruding portion 117 of the right eye camera 19 is disposed on the left side and the space between the right eye white light irradiation window 47 and the right eye excitation light irradiation window 51 is not used, the right eye camera 19 may not be disposed such that the imaging center 109 thereof is shifted downward along the first virtual line 105 with respect to the second virtual line 107. That is, the imaging center 109 of the right eye camera 19 may be disposed on the upper side with respect to the second virtual line 107, or may be disposed on the second virtual line 107. Similarly, since the protruding portion 117 of the left eye camera 21 is disposed on the left side and the space between the left eye white light irradiation window 49 and the left eye excitation light irradiation window 53 is not used, the left eye camera 21 may not be disposed such that the imaging center 109 thereof is shifted downward along the first virtual line 105 with respect to the second virtual line 107. That is, the imaging center 109 of the left eye camera 21 may be disposed on the upper side with respect to the second virtual line 107, or may be disposed on the second virtual line 107.

Although the embodiments have been described above with reference to the accompanying drawings, the present disclosure is not limited to such examples. It will be apparent to those skilled in the art that various changes, modifications, substitutions, additions, deletions, and equivalents can be conceived within the scope of the claims, and it should be understood that these changes, modifications, substitutions, additions, deletions, and equivalents also belong to the technical scope of the present invention. Components in the above-described embodiments may be optionally combined within a range not departing from the spirit of the invention.

It should be noted that the present application is based on a Japanese patent application (Japanese Patent Application No. 2019-002004) filed on Jan. 9, 2019, and the contents thereof are incorporated herein by reference.

### INDUSTRIAL APPLICABILITY

The present disclosure is useful as an endoscope capable of suppressing an increase of an outer diameter in an endoscope with two or more eyes.

### REFERENCE SIGNS LIST

15: endoscope

19: right eye camera

21: left eye camera

23: scope

29: rigid portion

37: transmission cable

39: image sensor

87: lens unit

91: imaging element holding member

101: distal end surface

103: axis line

105: first virtual line

107: second virtual line

109: imaging center

111: line segment

113: midpoint

115: cable accommodating portion

117: protruding portion

119: bent portion

121: adhesive material

1. An endoscope comprising:
a rigid portion provided at a distal end of a scope, which is formed in a substantially cylindrical shape and whose distal end surface has a substantially cylindrical shape; and
a plurality of cameras disposed on left and right sides of the rigid portion sandwiching a first virtual line orthogonal to an axis line of the rigid portion on the distal end surface,
wherein the plurality of cameras include a first camera, and the first camera is disposed such that an imaging axis is shifted from a second virtual line orthogonal to each of the axis line and the first virtual line in a direction along the first virtual line.

2. The endo scope according to claim 1, wherein
the plurality of cameras include a second camera, and
the first camera and the second camera are disposed such that a line segment connecting an imaging axes on the distal end surface is parallel to the second virtual line, and a midpoint of the line segment is separated from the axis line.

3. The endo scope according to claim 1, wherein
the first camera includes a lens unit coaxial with the imaging axis, and an imaging element holding member for positioning and fixing an image sensor to the lens unit.

4. The endo scope according to claim 3, wherein
the imaging element holding member has a cable accommodating portion for accommodating a transmission cable connected to the image sensor.

5. The endo scope according to claim 4, wherein
the cable accommodating portion includes a protruding portion that accommodates a bent portion of the transmission cable protruding from an outer shape of the image sensor in a direction orthogonal to the axis line.

6. The endoscope claim 5, wherein
the imaging axis is disposed to be shifted in a direction along the first virtual line in a direction opposite to a protruding direction of the protruding portion.

7. The endoscope according to claim 5, wherein
the bent portion is bent at an acute angle.

8. The endoscope according to claim 5, wherein
the bent portion is embedded in an adhesive material filled in the protruding portion.
